(19) United States
(12) Patent Application Publication
(10) Pub. No.: US 2008/0312531 A1
(43) Pub. Date: Dec. 18, 2008

(54) Apparatus and Method for Delivery of a Fluid or Sensor

(75) Inventors: Paul A. Campbell, Dundee (GB); Paul A. Prentice, Dundee (GB)

Correspondence Address: HESLIN ROTHENBERG FARLEY & MESITI PC, 5 COLUMBIA CIRCLE, ALBANY, NY, 12203, US

(73) Assignee: UNIVERSITY OF DUNDEE, Dundee (GB)

(21) Appl. No.: 12/066,170

(22) PCT Filed: Sep. 7, 2006
(86) PCT No.: PCT/GB2006/003323

§ 371 Date: Aug. 29, 2008

(30) Foreign Application Priority Data
Sep. 8, 2005 (GB) 0518270.4

(51) Int. Cl. A61M 37/00

(52) U.S. Cl. 600/431; 604/22

(57) ABSTRACT

An apparatus for the encapsulation and release or exposure of the contents of a chamber. The apparatus has a seal covering the opening to the chamber to encapsulate the contents of the chamber. The seal contains ultrasonically rupturable material which can be driven by an ultrasound signal to break the seal. The apparatus of the invention can be used for selective drug delivery and to contain a sensor, such as a micro-sensor, in a sealed environment prior to use.

The present invention relates to an apparatus and method for fluid delivery and in particular to an apparatus and method that allows for selective delivery of a medicament or a sensor.

Drug delivery technology has had a huge effect on modern medical practice, greatly improving the effectiveness of some drugs and also providing new applications for other drugs.

Drug diffusion systems allow sustained drug release patterns by diffusion through a polymer membrane or a bulk polymer. Reservoir devices generally consist of a semi-permeable barrier through which a drug is slowly released. In many cases, drug release is powered by electrical cells that have been implanted into the patient.

Coated bead and multi-particulate systems comprise pH sensitive, and/or sustained release coatings, with a large number of the beads or particles held in a capsule.

The beads or particles may deliver their contents in a sustained fashion, or upon contact with certain areas of the body.

It is an object of the present invention to provide improvements in and relating to the selective delivery of medicaments.

In accordance with a first aspect of the invention there is provided an apparatus for the encapsulation and release or exposure of the contents of a chamber, the apparatus comprising:
one or more chambers, one or more of the chambers having a seal covering the opening to the chamber to encapsulate the contents of the chamber, the seal containing
ultrasonically rupturable material;
wherein the ultrasonically rupturable material can be driven by an ultrasound signal to break the seal.

Preferably, the seal on one or more of said chambers comprises ultrasonically rupturable material having a characteristic response to the application of ultrasound such that each seal may be selectively ruptured.

Preferably, the selective rupturing of the seal occurs at the resonant frequency of the ultrasonically rupturable material.

Preferably, the seal further comprises a matrix in which the ultrasonically rupturable material is contained.

Preferably, the seal further comprises a binding agent.

Preferably, the binding agent is non-toxic and biocompatible.

Preferably, the ultrasonically rupturable material is distributed within the binding agent such that when the ultrasonically rupturable material is ruptured, voids or channels in the seal are created to break the seal. The creation of voids or channels can allow controlled release of the contents of the chamber.

Preferably, the ultrasonically rupturable material may be contained in the matrix, the ultrasonically rupturable material being driveable into sustained oscillation in response to an ultrasonic signal to cause a stress on the matrix which causes the contents of the chamber to be released from the chamber.

More preferably, the content of the chamber is released by means of a pumping action.

More preferably, at least part of the ultrasonically rupturable material is ruptured by the action of the ultrasound.

Preferably, the ultrasonically rupturable material is held in the interstices of the matrix.

Preferably the characteristic response is dependent upon ultrasound frequency and/or power and/or peak pressure.

Preferably, the ultrasonically rupturable material is one or more microbubble.

Preferably, the microbubbles are filled with a gas.

Preferably, the microbubbles are ultrasound contrast agents.

Preferably, the microbubbles have a diameter of less that 20 μm.

Preferably, the microbubbles have a diameter of between 1 and 10 μm.

Preferably, the microbubbles have an albumin shell.

Preferably, the microbubbles have a polymeric biocompatible shell.

Preferably, the apparatus further comprises one or more ultrasound source.

Preferably, the ultrasound sources are capable of emitting ultrasound within the frequency range of 0.02 to 30 MHz.

Preferably, the ultrasound sources are capable of emitting ultrasound at a peak negative pressure of 0.1 to 10 MPa.

Most preferably, the ultrasound source emits ultrasound at a peak negative pressure of 0.2 to 5 MPa.

Preferably, the ultrasound source emits ultrasound in pulses.

Preferably, the ultrasound pulses have a period of less than 250 ms.

Preferably, the ultrasound source is coupled to the microbubbles by means of a liquid.

Preferably, the liquid is a physiological medium.

Preferably, the apparatus is attached to a substrate.

Preferably, the apparatus is further provided with a tracer to assist in location thereof after it is delivered.

Preferably, the chamber contains an active component.

Preferably, the active component is a therapeutic agent.

Optionally, the active component is a reagent.

Optionally, the active component is a sensor.

Optionally, the sensor is a microfabricated sensor.

Preferably, a plurality of chambers are connected together.

Preferably, the plurality of chambers are formed from a single piece of material.

Optionally, the chambers may be connected by the ultrasonically rupturable material. The chambers may therefore be separated from one another by the application of ultrasound.

The arrangement of chambers can be shaped in any suitable configuration such as in a line with the openings arranged adjacent to one another, or with the chambers extending radially from a common connection point, or forming a three dimensional array having a shaped cross section which may be square or round or oval for example.

In accordance with the second aspect of the present invention there is provided a reservoir array having an active control mechanism which comprises the apparatus in accordance with the first aspect of the invention.

Preferably, the reservoir array is mounted on a substrate.

Preferably, the substrate is rigid.

In accordance with the third aspect of the invention there is provided a bolus incorporating the apparatus of the first aspect of the invention.

In accordance with a fourth aspect of the present invention there is provided a method for the release of the encapsulated contents of a chamber, the method comprising the step of:
applying an ultrasound signal to a seal on the chamber to rupture the ultrasonically rupturable material in the seal in order to release the contents of the chamber.

Preferably, the seal on one or more of said chambers comprises may be selectively ruptured by applying ultrasound at a characteristic frequency.

Preferably, the selective rupturing of the seal occurs at the resonant frequency of the ultrasonically rupturable material.

Preferably, the ultrasonically rupturable material may be contained in the matrix, the ultrasonically rupturable material being driveable into sustained oscillation in response to an ultrasonic signal to cause a stress on the matrix which causes the contents of the chamber to be released from the chamber.

More preferably, the content of the chamber is released by means of a pumping action.

More preferably, at least part of the ultrasonically rupturable material is ruptured by the action of the ultrasound.

Preferably the characteristic response is dependent upon ultrasound frequency and/or power and/or peak pressure.

Preferably, the ultrasound is emitted within the frequency range of 0.02 to 30 MHz.

Preferably, at a peak negative pressure of 0.1 to 10 MPa.

Most preferably, the ultrasound is emitted at a peak negative pressure of 0.2 to 5 MPa.

Preferably, the ultrasound is emitted in pulses.

Preferably, the ultrasound is emitted in pulses having a period of less than 250 ms.

Preferably, the method further comprises the steps of introducing the one or more chambers into a body and applying the ultrasound to the body.

The present invention will now be described by way of example only with reference to the accompanying drawings in which:

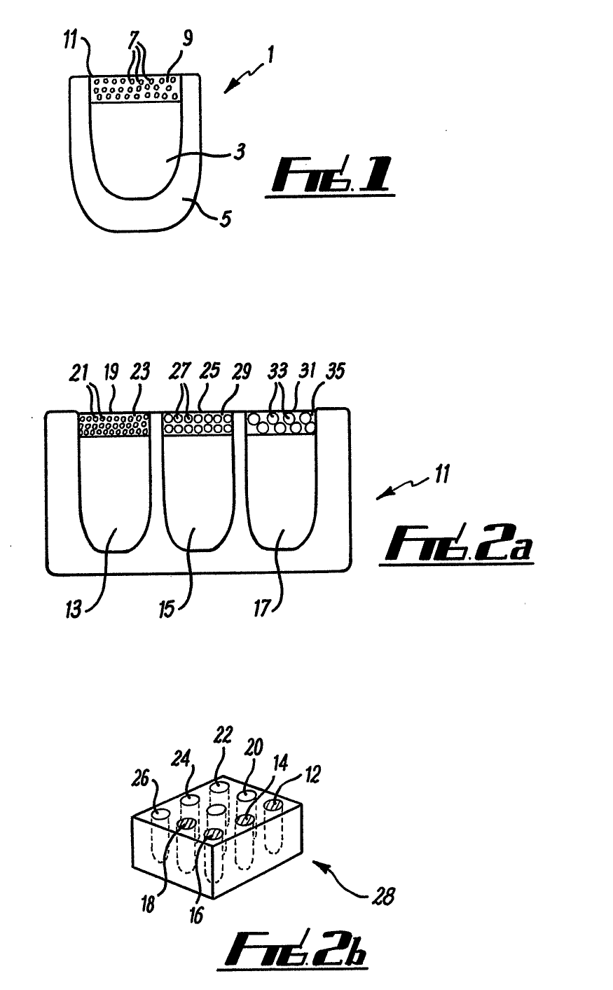

FIG. 1 is a schematic drawing of an apparatus of the present invention comprising one chamber;

FIG. 2a is a schematic drawing of an apparatus of the present invention comprising three medicament chambers, each sealed by a single microbubble and FIG. 2b shows a reservoir array;

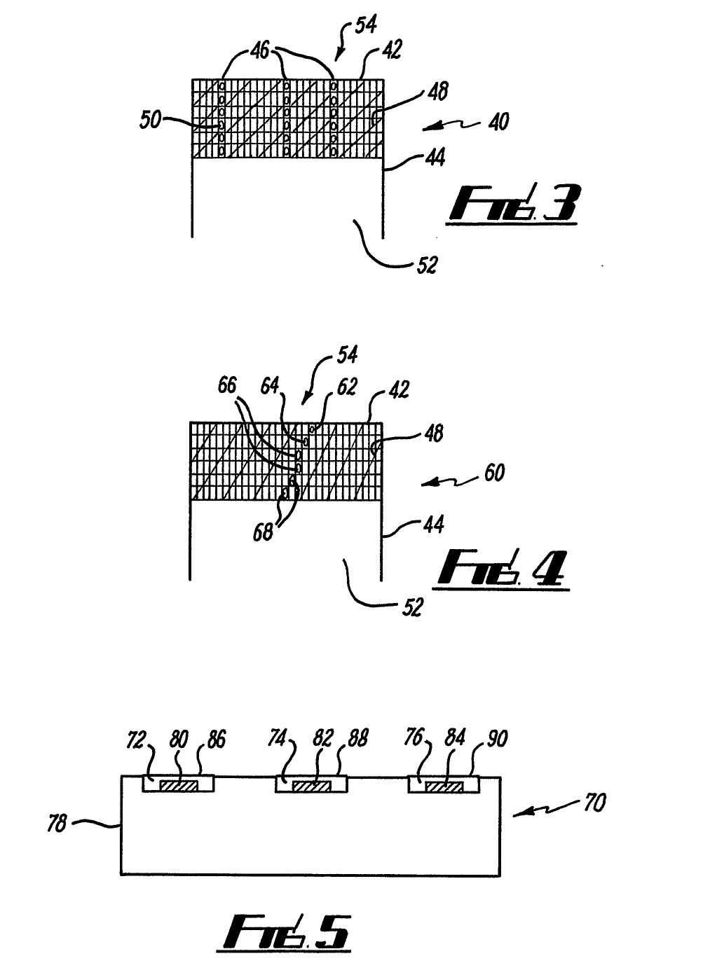

FIG. 3 is a schematic drawing of another embodiment of the present invention;

FIG. 4 is a schematic representation of another embodiment of the present invention; and

FIG. 5 is a schematic representation of a further embodiment of the present invention.

FIG. 1 shows an embodiment of the present invention in which the dispensing means 1 has a single chamber 3, sealed by means of microbubbles 7 embedded in a binder 9 and located across an opening 11. In this example of the present invention, the chamber 3 contains a therapeutic agent which may be a fluid or a number of microcapsules or microcaplets. The dispensing means is intended for controlled release of the medicament contained within the chamber, the medicament generally being a fluid although it could consist of a number of microcapsules or microcaplets.

The microbubbles 7 can be selectively ruptured by the application of a continuous ultrasound wave. This is achieved by use of an ultrasound source which transmits the continuous wave through the medium in which the dispensing means is located.

This medium is typically a liquid or an aqueous medium or any suitable medium that can effectively couple the ultrasound signal to the microbubbles. The microbubbles rupture in response to a specific ultrasound frequency.

In this way the delivery of a medicament to a patient can be controlled in response to time, or indeed in response to the location of the dispensing means in, for example, the digestive tract or the arterial system. The dispensing means may remain in vivo and be activated on demand.

In another embodiment of the present invention, the apparatus is provided with a seal formed from microbubbles contained in a binder, similar to those shown in FIG. 1 but in which the microbubbles are designed to rupture when a pulsed ultrasound signal is applied at a characteristic or resonant frequency.

FIG. 2a shows an apparatus means 11 comprising three chambers 13, 15 and 17, each having a microbubble seal 19, 25 and 31 respectively. The apparatus is designed for drug delivery in a body, in this example. Each chamber 13, 15 and 17 contains a different drug, and each microbubble seal 19, 25 and 31 is designed to rupture at a different frequency. Each seal comprises a plurality of microbubbles 21, 27 and 33 in a respective binder 23, 29 and 35. This enables control of delivery of each drug, whereby the characteristics of the incident ultrasound such as the frequency or power is tuned to selectively rupture the microbubbles 21, 27 or 33.

The apparatus can be used to deliver a specific drug as and when required. For example, it may be swallowed, and a first ultrasonic wave transmitted when it is in the oesophagus. This would release a first drug into the oesophagus. When it reaches the stomach, a second ultrasonic wave is transmitted which releases the second drug into the stomach. During the first and second drug delivery it is important that the drugs contained in chambers 15 and 17 are not released. Finally, when in the small intestine a third ultrasound wave is transmitted, and the third drug is released into the small intestine. In each release, the drug is delivered by the rupture of the respective microbubble in response to the ultrasonic signal at a characteristic frequency.

This allows for multi-stage drug delivery, actuable by a predefined ultrasonic frequency or power.

FIG. 2b shows another embodiment of the present invention in which chambers 12, 14, 16, 18, 20, 22, 24 and 26 are contained in a reservoir array 28. The array contains 9 reservoirs, each of which have openings to allow the contents of the reservoir to be released. In this example reservoirs 12, 14, 16 and 18 have caps that are rupturable upon the application of ultrasound, the remaining reservoirs have no caps. The apparatus of the present invention can be contained in a reservoir array in which some of the reservoirs have no caps or in which the contents of the reservoir is released by another mechanism.

FIG. 3 shows an embodiment of the present invention 40 in which the dispensing means comprises a matrix 42 positioned at or near the opening of a chamber 44. A binder 48 illustrated by diagonal shading is introduced and channels 46 are defined by the presence of microbubbles 50 at specific locations within the matrix. Upon activation of the ultrasound, the microbubbles are ruptured and allow an open channel to be created between the medicament containing chamber 52 and the outside of the chamber 54 thus releasing the medicament through the channels 46.

FIG. 4 shows a further alternative embodiment of the present invention. The dispensing means 60 comprises a matrix 42 adapted to accumulate microbubbles which have a range of ultrasound frequency responses. In this example, the matrix is arranged to have interstices which are sized to accept microbubbles of different sizes (62, 64, 66, 68). The microbubbles are arranged within the matrix such that when driven into sustained oscillation by the ultrasound, they impart a stress onto the matrix. The stress imparted to the matrix causes it to move in a controlled manner and to allow the fluid contained within the chamber to be released. The geometrical arrangement of the interstices assists in this process by prescribing the position of bubbles of different sizes thereby contributing to the controlled movement of the matrix.

It is also envisaged that such a dispensing means could remain in vivo by implantation and be actuated as and when required by the required ultrasonic signal in order to achieve a controlled release to local tissue.

FIG. 5 shows a further embodiment of the present invention in which three chambers 72, 74 and 76 are contained on a substrate 78. The chambers are provided by recesses, wells, holes or cavities formed in the substrates which may be a suitably physiologically tolerable material and may be rigid or pliable. The chambers 72, 74 and 76 contain a sensor for measuring a chemical, biochemical or physiological property of the area surrounding the apparatus. Typically the sensor would be microfabricated. Sensors 80, 82, 84 can be identical and be used for measuring a single feature of their surroundings or could measure a number of physical and or chemical characteristics.

In the example of FIG. 5, sensors 80, 82 and 84 have been microfabricated to measure a single physical property such as pH or chemical concentration. Each chamber 72, 74 and 76 has a cap 86, 88 and 90 which comprises an ultrasonically rupturable material. In addition, each cap is rupturable at a predetermined ultrasound frequency or power that is distinct from the ultrasound frequency or power that is required to rupture the other caps.

Therefore each of the sensors 80, 82, 84 can be selectively exposed to the surroundings by rupture of the cap. Advantageously, this allows each sensor to be enclosed in an inert or sterile environment until it is needed and avoids the well known problem of bio-fouling. Therefore, this example of the present invention provides a reliable, long term sensing implant.

Improvements and modifications may be incorporated herein without deviating from the scope of the invention herein intended. For example, medicament chamber seals might be effected by any number of microbubbles, each rupturable by any number of ultrasonic frequencies. Likewise, a chip may consist of any number of medicament chambers, to allow for any number of different medicaments, or any number of dispensing actions as a function of elapsed time.

1. An apparatus for encapsulation and release or exposure of contents of one or more chambers, the apparatus comprising:
one or more chambers, the one or more chambers each having a seal covering an opening to each chamber to encapsulate the contents of each chamber, the seal comprising ultrasonically rupturable material;
wherein the ultrasonically rupturable material can be driven by an ultrasound signal to break the seal.

2. An apparatus as claimed in claim 1, wherein the seal on one or more of said chambers comprises ultrasonically rupturable material having a characteristic response to an application of ultrasound such that the seal on the one or more chambers is selectively rupturable.

3. An apparatus as claimed in claim 2, wherein the selective rupturing of the seal occurs at a resonant frequency of the ultrasonically rupturable material.

4. An apparatus as claimed in claim 1, wherein the seal further comprises a matrix in which the ultrasonically rupturable material is contained.

5. An apparatus as claimed in claim 1, wherein the seal further comprises a binding agent.

6. An apparatus as claimed in claim 5, wherein the binding agent is non-toxic and biocompatible.

7. An apparatus as claimed in claim 5, wherein the ultrasonically rupturable material is distributed within the binding agent such that, when the ultrasonically rupturable material is ruptured, voids or channels in the seal are created to break the seal.

8. An apparatus as claimed in claim 4, wherein the ultrasonically rupturable material is contained in the matrix, and wherein the ultrasonically rupturable material is driveable into sustained oscillation in response to an ultrasonic signal to cause a stress on the matrix which causes the contents of the chamber to be released from the chamber.

9. An apparatus as claimed in claim 4, wherein the ultrasonically rupturable material is held in interstices of the matrix.

10. An apparatus as claimed in claim 2, wherein the characteristic response is dependent upon one or more of an ultrasound frequency, a powers and a peak pressure.

11. An apparatus as claimed in claim 1, wherein the ultrasonically rupturable material is one or more microbubbles.

12. An apparatus as claimed in claim 11, wherein the one or more microbubbles are filled with a gas.

13. An apparatus as claimed in claim 11, wherein the one ore more microbubbles are ultrasound contrast agents.

14. An apparatus as claimed in claim 11, wherein the one or more microbubbles have a diameter of less than 20 μm.

15. An apparatus as claimed claim 1, wherein the apparatus further comprises one or more ultrasound sources.

16. An apparatus as claimed in claim 15, wherein the one or more ultrasound sources emit ultrasound within a frequency range of 0.02 to 30 MHz.

17. An apparatus as claimed in claim 15, wherein the one or more ultrasound sources emit ultrasound at a peak negative pressure of 0.1 to 10 MPa.

18. An apparatus as claimed in claim 15, wherein the one or more ultrasound sources emit ultrasound in pulses.

19. An apparatus as claimed in claim 15, wherein the ultrasound pulses have a period of less than 250 ms.

20. An apparatus as claimed in claim 1, wherein the apparatus is further provided with a tracer to assist in locating the apparatus.

21. An apparatus as claimed in claim 1, wherein the chamber contains an active component.

22. An apparatus as claimed in claim 21, wherein the active component is a therapeutic agent.

23. An apparatus as claimed in claim 21, wherein the active component is a reagent.

24. An apparatus as claimed in claim 21, wherein the active component is a sensor.

25. An apparatus as claimed in claim 24, wherein the sensor is a microfabricated sensor.

26. An apparatus as claimed in claim 1, wherein a plurality of chambers is connected together.

27. An apparatus as claimed in claim 1, wherein a plurality of chambers is formed from a single piece of material.

28. An apparatus as claimed in claim 27, wherein the plurality of chambers is connected by ultrasonically rupturable material.

29. A reservoir array having an active control mechanism which comprises the apparatus in accordance with claim 1.

30. A reservoir array as claimed in claim 29, wherein the reservoir array is mounted on a substrate.

31. A reservoir array as claimed in claim 30, wherein the substrate is rigid.

32. A bolus incorporating the apparatus of claim 1.

33. A method for release of encapsulated contents of a chamber, the method comprising:
applying an ultrasound signal to a seal on the chamber to rupture ultrasonically rupturable material in the seal in order to release the contents of the chamber.

34. A method as claimed in claim 33, wherein applying the ultrasound signal comprises selectively rupturing the seal on one or more of the chambers by applying ultrasound at a characteristic frequency.

35. A method as claimed in claim 34, wherein selectively rupturing of the seal occurs at a resonant frequency of the ultrasonically rupturable material.
